(19) United States
(12) Patent Application Publication
(10) Pub. No.: US 2026/0243712 A1
(43) Pub. Date: Aug. 20, 2026

(54) QUANTIFYING IMMOBILE AND PRODUCIBLE RESERVOIR FLUIDS

(71) Applicant: CONOCOPHILLIPS COMPANY, Houston, TX (US)

(72) Inventors: Tianmin JIANG, Houston, TX (US); Hesham EL-SOBKY, Houston, TX (US)

(73) Assignee: CONOCOPHILLIPS COMPANY, Houston, TX (US)

(21) Appl. No.: 19/529,511

(22) Filed: Feb. 4, 2026

Related U.S. Application Data
(60) Provisional application No. 63/760,971, filed on Feb. 20, 2025.

(51) Int. Cl. G01N 24/08; G01N 15/08; G01N 33/24

(52) U.S. Cl. CPC G01N 24/081; G01N 15/088; G01N 33/241

(57) ABSTRACT

A workflow for data acquisition and processing to quantify immobile and producible fluids more accurately from log and core measurements is described. Also described are methods of reservoir characterization to distinguish producible from immovable fluids that use NMR measurements on core samples as endpoint references and calibration points for downhole logging. Endpoint signatures of pore fluids are easily identified by the NMR of core samples, thus providing more accurate results on formation fluid porosities and saturations from measured data.

### PRIOR RELATED APPLICATIONS

This application claims priority to U.S. Ser. No. 63/760,971, filed Feb. 20, 2025, and incorporated by reference in its entirety for all purposes.

### FEDERALLY SPONSORED RESEARCH STATEMENT

Not applicable.

### FIELD OF THE DISCLOSURE

The disclosure generally relates to workflow and methods for core analysis and well logging. More particularly, the disclosure provides a new way of correcting for high smectite content and accurately distinguishing between mobile and immobile fluids.

### BACKGROUND OF THE DISCLOSURE

There is an ever increasing demand for energy due to improving standards of living, advancements in technology, and high energy requiring technology, such as AI databases and cryptocurrencies. The drive for energy leads to more green energy development, but also more hydrocarbon development. In fact, hydrocarbon exploration is moving to more challenging geological settings and thus systematic methods for the optimization of oil production are increasingly required to meet the global energy demand. Consequently, the use of integrated approaches of geology, geophysics, petrophysics, geostatistics and reservoir engineering for detailed characterization of reservoirs and their properties is crucial to meet demand.

Reservoir characterization is a collection of techniques that deal with quantifying rock and fluid properties (porosity, permeability and hydrocarbon saturation) of a reservoir. These techniques refer to all the relevant information that is required to describe a reservoir in terms of its ability to store and produce hydrocarbons. This includes understanding the architecture of the reservoir, such as its internal and external geometry, its static properties (distribution of reservoir properties such as porosity, permeability and net pay thickness) as well as dynamic properties (understanding the fluid flow within the reservoir) models. This information can be used to predict the future performance of reservoirs, improve the rate of production, revitalize oil fields, and helps to build accurate financial models for oil companies.

Traditional static models for formation evaluation are based on combined datasets. Density, neutron, resistivity and gamma ray logging while drilling or logging while tripping have been widely used to characterize reservoir formations and provide cost-effective estimates of lithology, total porosity and water saturation. Nevertheless, actual results in production may be quite different from predicted results, largely because these static models cannot distinguish immovable fluids from producible fluids.

Recent developments in simultaneous acquisition of nuclear magnetic resonance (NMR) do allow the user to distinguish immobile from mobile fluids. Using relaxation time in longitudinal (T₁) and transverse (T₂) directions we can create an intensity map of T₁-T₂ relaxation time distributions. This NMR T₁-T₂ log data shows unique signatures of formation fluids, such as gas, immobile hydrocarbons and water, producible oil and water. It can also provide fluid and matrix properties, including fluid viscosity, pore geometry and fluid-pore interaction.

However, in NMR logging, due to the downhole environment and the resolution limitations of the logging tool, the log NMR data usually has relatively low signal-to-noise ratio (SNR) and the signatures of the fluids are not always well-separated. Therefore, it may be challenging to visually separate the signal contributions of different formation fluids on T₁-T₂ maps based on downhole NMR.

Yet another method that allows us to at least partially distinguish mobile from immobile fluids is retort—heating to drive off fluids. Retort methods have been widely used to get oil and water fluid saturations by a high-temperature retorting process in which the oil and water contained in a fresh sample of crushed core material are vaporized, condensed, and collected in calibrated glassware (Recommended Practices for Core Analysis, 1998, available online at energistics.org/sites/default/files/2022-10/rp40.pdf).

However, traditional open retort measurements on crushed core materials have two obvious limitations: 1) the rock crushing process causes fluid loss, especially for rock samples with abundant producible/mobile fluids; 2) the material container in open retort system is not sealed and open to atmosphere, resulting in fluid loss due to evaporation. Thus, while an improvement, retort methods could be made more accurate.

Thus, what is needed in the art are better methods of reservoir characterization that are able to distinguish producible from immovable fluids and thus provide better predictions, especially in reservoirs with significant amounts of immovable fluids. The ideal method would provide one or more workflows and methods that are fully compatible with existing technology, be cost effective, and provide better predictions of actual reservoir behavior. This invention meets one or more of these needs.

### SUMMARY OF THE INVENTION

To address and solve the issue of low signal-to-noise ratio (“SNR”) in downhole NMR logging runs, laboratory benchtop NMR measurements on core samples are obtained and used as endpoint references and calibration points for downhole logging (“log NMR”), as well as all of the other traditional methods of obtaining and using reservoir data. NMR of core samples (“core NMR”) has much better SNR than log NMR and can easily show endpoint T₁-T₂ signatures of pore fluids. The T₁-T₂ signatures of separated fluids can thus be used to characterize fluid mobility, pore sizes and formation wettability and together with other data, will provide improved reservoir predictions.

In addition to NMR, thermal extraction via closed retort is implemented herein to separate pore fluids with different mobilities, but in this case closed retort is used, not traditional open retort, and the temperature profile may be modified as needed. During the closed retort process, a core sample is heated in a closed steel container, and all emitted fluids are captured for analysis. In these experiments, pore fluid with different mobilities are released at different temperatures. With proper temperature cutoffs, clay-bound water, matrix pore water and hydrocarbon are easily separated and quantified.

For rock samples that lack smectite—a hydrous silicate of alumina, of a greenish color, which, in certain states of humidity, appears transparent and almost gelatinous—the closed retort method is accurate for pore fluid typing and quantification. However, if the rock contains smectite or other clays, clay-bound water associated with smectite (clay-bound water associated with other type of clays usually don't have this issue) may release at lower temperatures, where producible water in the matrix can also get released, resulting in an underestimate of clay-bound water and an overestimate of producible water.

To overcome the interference in the closed retort quantification of clay-bound water, we propose that H₂O/D₂O exchange is implemented to distinguish immobile water from producible water and can also be used to confirm NMR T₁-T₂ data and closed retort data. Deuterium has no ¹H NMR response, making it invisible to ¹H NMR measurement. Thus, time-lapse NMR measurements on H₂O/D₂O exchange process give the evolution of residual water in rock sample, separating immobile water (which has an NMR signal) from producible water (which has a different NMR signal in D₂O) in the appropriate timeframe. The time-lapse T₂ distribution of immobile water shows bimodal behavior, with one peak including clay-bound water and the other including capillary-bound water. A proper T₂ cutoff can then be used to separate clay-bound water from capillary-bound water.

This data can be used to correct both NMR T₁-T₂ and closed retort data. The disclosure thus addresses the limitations of static models and the inability to distinguish producible from immobile fluids by providing methods to more accurately quantify immobile and producible fluids in the formation, as summarized in FIGS. 1A and 1B. These data can then be combined with traditional data in reservoir modelling to improve predictive results, especially for smectite-rich plays.

Generally speaking, core samples are obtained, in any manner known in the art. “Recommended Practices for Core Analysis,” incorporated by reference in its entirety for all purposes, describes many techniques for collecting and analyzing core samples.

The core sample is first trimmed (See FIG. 1A) to remove any surface defects or contamination and to provide a regular shape (typically a cylinder). The remaining core is split e.g., lengthwise into two halves [101]. The halves may be subdivided as needed to provide intact portions of the core for each of any analyses that require intact core, but where a first analysis does not impact a follow-on analysis (as described herein), the same core sample can be used for more than one test.

Bulk volume (BV) is ascertained at some point, either of the core whole or the portions thereof, as desired. BV can be measured by Archimedes (buoyancy) mercury immersion and the same sample can be used for other tests if no mercury enters the core. Caliper calculation to measure the volume of a cylinder before halving is preferred, but other methods can also be used.

Bulk volume (V_{b}) minus grain volume (V_{g}) (see below) provides one measure of total porosity. Another method of providing total porosity is to crush, solvent extract, and dry the sample. If the sample is dried and then weighed, the dry bulk density can be used to calculate porosity by assuming the air filling the pores to have a fluid density of 0 g/cm³. This can be done on tested samples as shown in FIG. 1 but can also be done with a separate portion or sample of core or even with trimmings.

Other standard tests may also be performed, such as saturation, permeability, petrographic characterization, grain size distribution, oil gravity, and characterization, acid solubility, core water salinity determination, and the like. However, these are not detailed herein since we focus instead on the various tests needed to correctly differentiate mobile from immobile fluids.

Total organic carbon (TOC) and Rock-Eval pyrolysis measurements, as well as core mineralogy are performed on the trim remnants [102]. TOC can be by any method known in the art, including LECO method (ISO 10694:1995) and/or the Rock-Eval pyrolysis method, which can provide further detail about carbon content, separating hydrocarbons with differing mobilities. We have used LECO equipment and Rock-Eval pyrolysis methods of obtaining TOC.

In Rock-Eval pyrolysis, a sample is placed in a vessel and is progressively heated to temperature (about 550° C.) under an inert atmosphere. During the analysis, the hydrocarbons already present in the sample are volatized at a moderate temperature. The amount of hydrocarbons are measured and recorded as a peak known as S1. Next pyrolyzed is the kerogen present in the sample, which generates hydrocarbons and hydrocarbon-like compounds (recorded as the S2 peak), CO₂, and water. The CO₂ generated is recorded as the S3 peak. Residual carbon is also measured and is recorded as S4.

The core mineralogy measurement can be done in any method known in the art, such as XRD, EDX, XRF, SEM, LPSA, FTIR, ICP-MS, and thin sectioning. We have used XRD (Burnett A D, 1995) or FTIR (ASTM E1252-98) herein. Core minerology provides the weight fractions of inorganic minerals and is used to determine whether the rock sample is smectite-rich.

The workflow is designed mainly for unconventional rock (organic shale or tight rock). We prefer using 2 half plugs as they provide duplicate results and we need not await smectite levels before proceeding. In addition, in many cases, the rock sample is not homogeneous, and if the gas or liquid porosities from the two half plugs are inconsistent, and because of the lack of homogeneity, the workflow is discontinued. However, if preferred, the samples may be further subdivided, or the analysis may be simplified to avoid the duplicates. Both approaches are described herein.

In FIG. 1A porosities are measured on both core halves [103,110] by any method known in the art, for example, gas-driven solvent extraction, gas injection porosimetry (GIP), Barnes method, helium porosity, bulk volume (V_{b}) minus grain volume (V_{g}), stereology, radiation scattering, pycnometry, mercury intrusion porosimetry (MIP), water immersion porosimetry (WIP), gas expansion (GE), gas expansion induced water intrusion porosimetry (GEIWIP), adsorption from the gas phase, intrusion, suction, fluid flow, and ultrasonic methods.

We have used the GIP method using helium herein as an initial porosity determination. GIP provides total gas accessible porosity, assuming that the compressibility of the water and/or oil present in the other pore spaces is minimal. When GIP is used, the same sample can be subjected to the next test, as liquids in the core are unchanged by this test. Thus, the entire core half can be tested.

NMR is also measured on both core halves [104,111] to obtain endpoint T₁-T₂ signatures of pore fluids by any method known in the art. T₁-T₂ refer to the relaxations parallel (T₁) and perpendicular (T₂) to the external magnetic field. NMR doesn't change the fluids in the core, so the same core sample may be used in certain follow-on tests. (Dick et. al., 2022).

The next step is closed retort on the gas-tested and NMR-tested half plug 1. Based on the mineralogy data [102], if the rock sample has little or no smectite [105], a two-temperature-step closed retort measurement is performed on the core sample to quantify clay-bound and matrix pore water (including producible water and capillary-bound water), and light oil. Step-one temperature (Temp 1) is selected to separate released water into clay-bound and matrix pore water. Step-two temperature (Temp 2) is set at the point that all the water and light oil get released from the plug. Temp 1 and Temp 2 may vary with the rock and the fractions to be quantified, but here we set Temp 1 at 130° C. (see FIG. 4). The evaporation of light components is about 150-410° C. so this is a suitable Temp 2 range. Heavy components begin cracking at 410-550° C. See “Recommended Practices for Core Analysis” for the methodology.

On the other hand, if the rock is smectite-rich, it is hard to differentiate clay-bound water and matrix pore water using thermal extraction method. In this case, a single-temperature (at Temp 2) closed retort measurement may be performed on samples of the core to get light oil and total water porosities. As indicated in FIG. 1A, both temperature profiles may be used—one on each half.

After the closed retort measurement, an intact portion of half-plug 1 or the prior tested sample(s) or the trimmings are crushed, solvent-cleaned and oven-dried to obtain grain volume and total porosity [107 or 109]. Total porosity minus the light oil and total water porosities from the closed retort test then provide porosity of immobile hydrocarbons.

For half-plug 2, by contrast, after NMR a H₂O/D₂O (water and heavy water) exchange experiment [112] is performed to obtain the relative proportions of diffusible (producible water) to non-diffusible water (clay-bound and capillary-bound water) in the rock. The time-lapse core NMR measurement is used to monitor the evolution of H₂O/D₂O exchange process [112]. As the D₂O diffuses into the core, replacing producible water, its signal disappears, leaving only immobile water signals (see URTeC-5015).

Following H₂O/D₂O exchange experiment, closed retort and post-retort experiments may be performed on half plug 2 to obtain different portions of fluid porosities, using similar procedures as used for half plug 1, but applying the single Temp 2 closed report profile.

Core data obtained as described herein plus log NMR T₁-T₂ data can be used together to improve pore fluid typing and quantification over log NMR analysis alone. Fluid porosities from T₁-T₂ data [104, 111, 115] are integrated and calibrated with those from closed retort and post retort analysis [106/107 or 108/109 and 113, 114] as well as H₂O/D₂O exchange experiments. The cross-validated and calibrated data of pore fluids from multi-physics give the final answer of immobile and producible water and hydrocarbons (see SPWLA-2015-SSS).

FIG. 1A supposes that the core sample is cut in half and certain assays are duplicated to provide duplicate results. Although possibly repetitive, providing both tracks regardless of smectite content allows work to proceed quickly without awaiting smectite results, plus the data redundancy confirms the accuracy of the results. However, if time permits or if samples are limited, one may await smectite results and only switch to single temperature closed retort and H₂O/D₂O exchange when the smectite results confirm the need for these changes. This workflow is shown in FIG. 1B. Here, the workflow is similar to that in FIG. 1A, but in this instance, we await smectite results and only then take the right (dotted arrows) or left (solid arrow) paths depending on smectite levels. If duplicate or triplicate results are desired, the cores can be subdivided, or additional core samples from the same play assayed. Further, the order of GIP and NMR can be swapped.

FIG. 2 shows the various porosities and how they are obtained. Bulk volume 217 is assessed and gives the total volume of the core (fluids plus solids). The core contains gas filled pores measured by GIP 201. Producible and capillary-bound water volume are measured by closed retort at Temp 1 and Temp 2 203, 204, if there is no significant smectite 202. If there is significant smectite 209, which case the closed retort is done at the higher temperature Temp 2 211. Producible oil is determined by closed retort at Temp 2 205, 210. NMR work also helps to determine the various porosities and/or amounts of the various fluids. NMR T₁-T₂ ascertains total porosity 212 and in some cases can distinguish the various fluids. However, D₂O exchange is more reliable for distinguishing producible water porosity 213 and capillary-bound water porosity 215 from clay-bound water porosity 214. The porosity summation of all released fluids (including light oil and all the water) during retort test gives retort porosity 206. Bulk volume BV 217 and post retort total grain volume V_{g} 207 confirm the value for total porosity 207. The difference between this total porosity and retort porosity provides porosity of immobile hydrocarbon 208.

In H₂O/D₂O exchange experiment, producible water 213 can be separated from immobile water by diffusion. Among immobile water, clay-bound water 214 can be separated from capillary-bound water 215 from time-lapse T₂ distribution of the rock sample with a proper T₂ cutoff.

The invention includes any one or more of the following embodiments, in any combination(s) thereof:
A method comprising a) obtaining a sample of core from a reservoir; b) trimming said core to produce trimmings and a remnant of core (hereinafter core); c) soaking said core in D₂O and performing time-lapse NMR on H₂O/D₂O exchange in said D₂O soaked core to quantify clay-bound water volume in said core.
A method comprising a) obtaining a sample of core from a reservoir; b) acquiring simultaneous nuclear magnetic resonance (NMR) relaxation time in longitudinal (T₁) and transverse (T₂) directions (NMR T₁-T₂) on said core to determine an amount of matrix pore water and an amount of clay-bound water; c) soaking said core in D₂O; d) performing time-lapse NMR on H₂O/D₂O exchange in said D₂O soaked core to quality check said NMR T₁-T₂, wherein said H₂O/D₂O exchange eliminates a matrix pore water signal, leaving a clay-bound water signal; and e) using said clay-bound water signal from step d) to correct said amount of clay-bound water from step b).
A method comprising a) obtaining a sample of core from a reservoir; b) trimming said core to produce trimmings and a remnant of core; c) obtaining gas injection porosity from a portion of said core; c) acquiring simultaneous nuclear magnetic resonance (NMR) relaxation time in longitudinal (T₁) and transverse (T₂) directions (NMR T₁-T₂) from a portion of said core; d) obtaining core minerology data from said trimmings to determine a level of smectite in said core and performing steps f) and i) if said trimmings have 0 to <5% smectite, but performing steps g), h), and i) if said trimmings have ≥5% smectite; c) obtaining closed retort data from a portion of said core at Temp 1 and Temp 2, wherein Temp 1 is a temperature where mobile water is emitted from said core and Temp 2 is a temperature where bound water and light oils are emitted from said core; or d) soaking a portion of said core in D₂O and performing time-lapse NMR on H₂O/D₂O exchange in said D₂O soaked core to separate and quantify producible water from immobile water; e) obtaining closed retort data on a portion of said core at Temp 2; and f) integrating core data to separate and quantify producible water, capillary-bound water and clay-bound water, and to separate and quantify producible hydrocarbons and immobile hydrocarbons.
A method comprising a) obtaining a sample of core from a reservoir; b) trimming said core to produce trimmings and a remnant of core and dividing said core into at least half-1 and half-2; c) core minerology data from a first portion of said trimmings to determine a level of smectite in said core and obtaining Rock-Eval total organic carbon (TOC) from a second portion of said trimmings; d) gas filled porosity from said half-1 and said half 2 to obtain a gas volume in said core; e) obtaining simultaneous nuclear magnetic resonance (NMR) relaxation time in longitudinal (T₁) and transverse (T₂) directions (NMR T₁-T₂) from said half-1 and said half 2 to quantify volume(s) of one or more of producible water, capillary-bound water, clay-bound water, producible hydrocarbons, and immobile hydrocarbons; f) obtaining closed retort data on said half-1 to quantify volume(s) of one or more of producible water, capillary-bound water, clay-bound water, and producible hydrocarbons at Temp 1 and Temp 2 if said core has no smectite, wherein Temp 1 is a temperature where producible water and capillary-bound water are emitted from said core and Temp 2 is a temperature where clay-bound water and light ends are emitted from said core or at Temp 2 if said core has significant smectite (≥5%); g) soaking said half-2 in D₂O and performing time-lapse NMR on H₂O/D₂O exchange in said D₂O soaked half-2 to obtain a volume of clay-bound water; h) obtaining closed retort data at Temp 2 on said soaked half-2 to obtain a volume of total water and a volume of producible oil; i) crushing and cleaning and drying a portion of said half-1 from step f) and said soaked core half-2 from step h) to obtain a total fluids volume in said core; and j) integrate half-1 and half-2 data from steps d) through i) using multiphysics simulations to separate and quantify a volume of: producible water, capillary-bound water, and clay-bound water and to separate and quantify a volume of immobile hydrocarbons and producible hydrocarbons and using S2 from said Rock-Eval TOC as reference in comparison to immobile hydrocarbon porosity.

Core data (or half-1 and half-2 data) includes the various types of fluid volume data obtained from any of the performed steps. TOC, Smectite, Rock eval, and the like, contribute to our overall understanding of the core, and are useful, but not essential for determining fluid volumes.

Any method herein described wherein bulk volume of said core is determined and used to convert volumes to porosity.

As used herein, the term “light oil” means C₆-C₂₀₊ hydrocarbons, but excluding kerogen.

As used herein, reference to a “portion” of a core sample, may mean the entire sample, a half sample or further subdivided samples. The method can be practiced on the same sample or half samples if the order proceeds as shown, but if apportioned, the different analyses may proceed at the same time or in any order.

As used herein “multiphysics simulation” (often shortened to simply “multiphysics”) is defined as the simultaneous simulation of different aspects of a physical system or systems and the interactions among them. We use multiphysics herein to combine data from various methodologies, improving overall accuracy. Any suitable multiphysics package may be used in the integration process, and we used an internal proprietary program herein.

As used herein, “producible oil” is the oil that can be obtained from the rock by heating to the requisite temperature, typically about 300° C. This is contrasted by non-producible hydrocarbons that will not be produced at that temperature but will typically be degraded or chemically changed if even higher temperatures are attempted.

As used herein, “matrix pore water” is the water that can be obtained from the rock by heating to the requisite temperature, typically about 120-140° C. Matrix pore water includes producible water and capillary-bound water (not clay bound water).

As used herein, “producible water” is the water that can be produced during production.

As used herein, “capillary-bound water” is the water bound by capillary forces. Capillary-bound water is part of immobile water, which cannot be displaced in H₂O/D₂O exchange experiments.

As used herein, “clay-bound water” is the water that is bound by clays. Clay-bound water associated with smectite may be released at lower temperatures, resulting in an underestimate of clay-bound water and an overestimate of producible water. Clay-bound water cannot be displaced in H₂O/D₂O exchange experiments.

As used herein, “immobile water” is clay-bound water and capillary-bound water.

As used herein, “bound fluid” is immobile water plus immobile hydrocarbon (immobile HC).

As used herein “porosity” determines reservoir storage capacity. It is defined as the ratio of void space, commonly called pore volume, to bulk volume and is reported either as a fraction or a percentage. Almost all hydrocarbon reservoirs are composed of sedimentary rocks in which porosity values generally vary from 10 to 40% in sandstones and from 5 to 25% in carbonates.

As used herein “total water porosity” is the sum of pore volume occupied by producible water plus capillary-bound water plus clay-bound water over bulk volume.

As used herein, producible fluid porosity or φ_{producible} is the volume of pores occupied by any producible water and producible hydrocarbon over bulk volume.

As used herein “retort porosity” means the level of porosity as determined by closed retort methods—a type of summation of fluids measurement wherein the amount of hydrocarbon, producible water, capillary-bound water and clay-bound water are determined by driving these fluids off at increasing temperatures and measuring their volume or weight. As noted herein, water volumes may be inaccurate in high smectite samples, but this can be corrected by analyzing D₂O/H₂O exchange.

As used herein “total porosity” or φₜ includes all void space regardless of whether the pores are interconnected or isolated. There is no practical way in the laboratory to measure isolated pore volume routinely on rocks. However, it can be determined by disaggregating the samples. Herein we determined the total porosity by crushing and measuring V_{g} and subtracting from bulk volume. φₜ=(BV−V_{g})/BV.

As used herein “grain volume” or V_{g} means the volume of solid material in the core sample as determined by crushing the sample and placing the resulting rock grains into a pycnometer or graduated cylinder along with a known volume of liquid. The volume of the rock grains can then be determined from the apparent volume change of the liquid (the Russell Method) after immersion or the apparent weight change (the Melcher-Nutting Method) of the immersed sample due to buoyancy using Archimedes' Principle.

As used herein “gas filled porosity” refers to the porosity as determined by gas injection porosimetry, as described by Sun (2016) or any roughly equivalent method. Since only interconnected gas-containing pores contribute to this measurement, it provides effective porosity or de. However, core analysis experience indicates that for most reservoir rocks there are few isolated pores, and hence there is very little or no measurable difference in historically defined effective and total porosity.

As used herein, “bulk volume” or BV is the total volume of the core—pores plus solids. It can be determined by one of two methods, physical measurement and displacement. The use of physical measurements is only applicable to core samples with regular geometric shape. For displacement, the core is placed in liquid mercury and the volume change recorded.

The use of the word “a” or “an” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim. The phrase “consisting of” is closed, and excludes all additional elements. The phrase “consisting essentially of” excludes additional material elements, but allows the inclusions of non-material elements that do not substantially change the nature of the invention, such as instructions for use, solvents, lab equipment, and the like.

Any claim or claim element introduced with the open transition term “comprising,” may also be narrowed to use the phrases “consisting essentially of” or “consisting of,” and vice versa. However, the entirety of claim language is not repeated verbatim in the interest of brevity herein.

The following abbreviations are used herein:

| ABBREVIATION | TERM (Cites herein incorporated by reference in its entirety for all purposes) |
|---|---|
| BV | Bulk volume |
| CPMG | Carr-Purcell-Meiboom-Gill |
| EDX | Energy-dispersive X-ray spectroscopy |
| FID | free induction decay |
| FTIR | Fourier-transform infrared spectroscopy |
| GE | Gas expansion |
| GEIWIP | Gas expansion induced |
| GIP | Gas injection porosimetry water induced porosimetry |
| ICP | Inductively coupled plasma |
| ICP-MS | Inductively coupled plasma mass spectrometry |
| LPSA | Laser particle size analysis |
| MIP | Mercury induced porosimetry |
| MS | Mass spectrometry |
| NMF | Non-negative matrix factorization |
| NMR | Nuclear magnetic resonance |
| NMR T₁-T₂ | The decay of RF-induced NMR spin polarization is characterized in terms of two |
| | separate processes, each with their own time constants. One process, called T₁, |
| | is responsible for the loss of resonance intensity following pulse excitation. The |
| | other process, called T₂, characterizes the width or broadness of resonances. |
| | Stated more formally, T₁ is the time constant for the physical processes |
| | responsible for the relaxation of the components of the nuclear spin |
| | magnetization vector M parallel to the external magnetic field, B0 (which is |
| | conventionally designated as the z-axis). T2 relaxation affects the coherent |
| | components of M perpendicular to B0. In conventional NMR spectroscopy, T₁ |
| | limits the pulse repetition rate and affects the overall time an NMR spectrum can |
| | be acquired. Values of T₁ range from milliseconds to several seconds, depending |
| | on the size of the molecule, the viscosity of the solution, the temperature of the |
| | sample, and the possible presence of paramagnetic species (e.g., O2 or metal |
| | ions). |
| PV | Pore volume |
| SEM | Scanning electron microscopy |
| SNR | Signal to noise ratio |
| TOC | Total organic carbon-usually determined by the acid digestion method or the Rock- |
| | Eval pyrolysis method wherein rocks are heated with a programmed temperature |
| | profile in a pyrolysis oven in an inert atmosphere (helium) of a small sample (~100 |
| | mg) to quantitate rock fluids. See e.g., leco.com/news/total-organic-carbon-with- |
| | two-methods/See also IFPEN-160000, IFPEN-300000, IFPEN-310000, IFPEN- |
| | 320000, IFPEN-400000 and IFPEN-500000 |
| WIP | Water induced porosimetry |
| XRD | X-ray diffraction |
| XRF | Xray Fluorescence |

### BRIEF DESCRIPTION OF THE DRAWINGS

FIG. 1A. Workflow for quantifying immobile and producible reservoir fluids with integrated measurements and tests. This workflow splits a core in half, and analyzes each half very similarly, though only one half is subject to D₂O exchange, and the retort temperature profile varies.

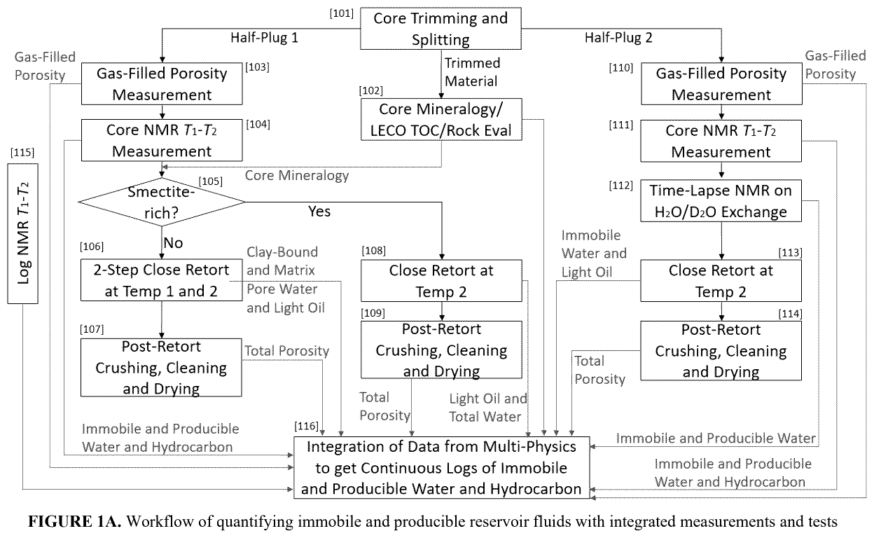

FIG. 1B. A simplified workflow for NMR data correction. The workflow is indicated by thin solid or dotted arrows and data collection is indicated by hatched arrows. Here, D₂O exchange and the retort temperature at Temp 2 are only done if there is high smectite.

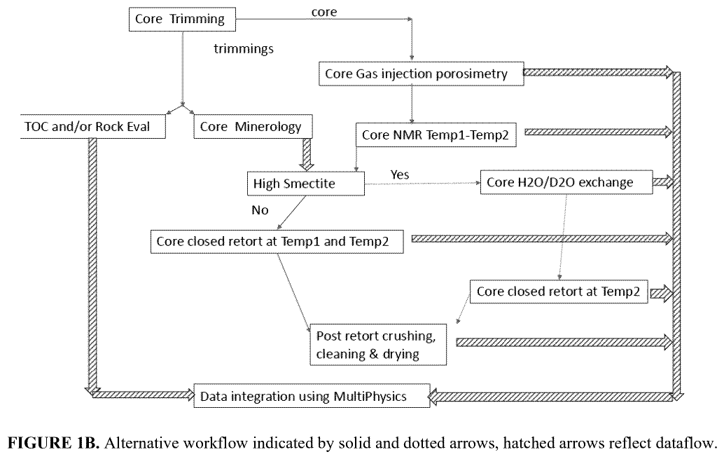

FIG. 2. Schematic illustration of various fluid porosities obtained from different core measurements.

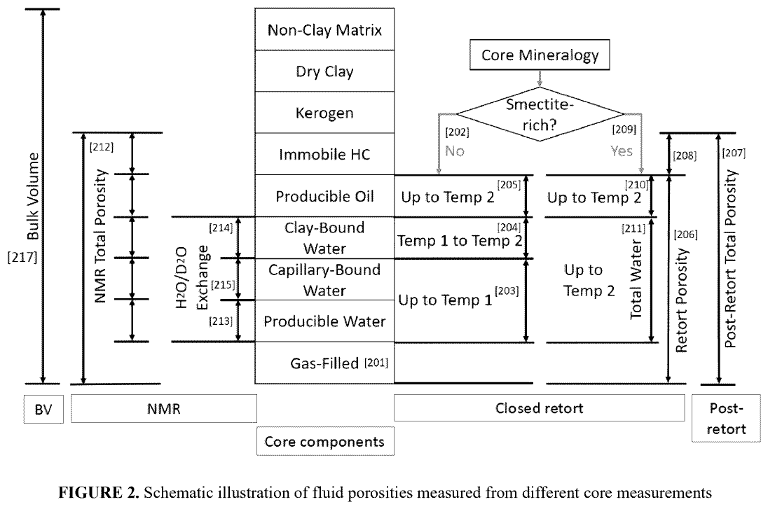

FIG. 3. NMR T₁-T₂ map with cutoff lines for fluid typing and quantification.

FIG. 4. Incremental water released (3 samples) along retort temperature profile exhibits two distinct peaks interpreted as differentiation of clay-bound and matrix pore water.

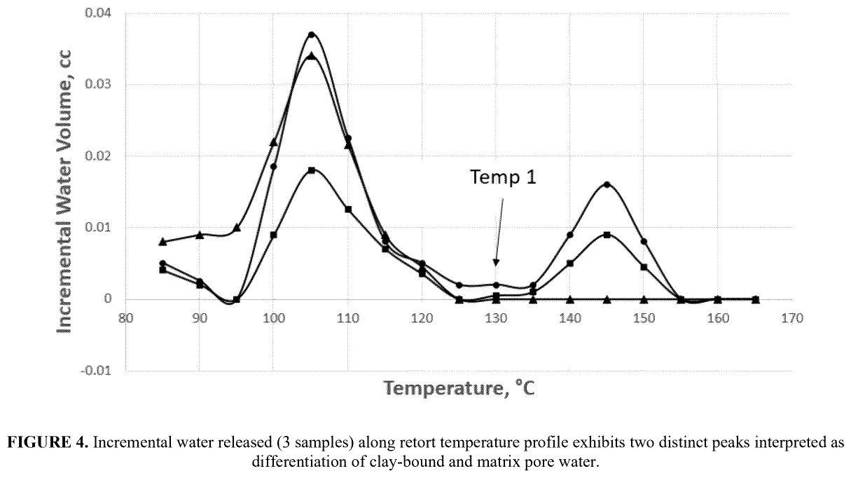

FIG. 5. Time-lapse evolution of NMR T₂ distribution. Track 1: Time point of NMR acquisition; Track 2: T₂ distribution of rock sample and effluent at each acquisition time; Track 3: T₂ distribution of the rock sample with T₂ cutoff for bound fluid. Track 3 shows the porosities of remaining fluid (solid curve) and plateau value (black solid line) at time end, as well as bound fluid porosity (dotted curve) and the average value (gray solid line).

FIG. 6. Time-lapse evolution of NMR porosity of the remaining fluid in rock sample. Sample 1 curve: NMR porosity evolution reaches a plateau at time end; Sample 2 curve: NMR porosity evolution doesn't reach a clear plateau at time end. The NMR porosity of remaining fluid at time end is residual NMR porosity. The difference between initial and residual NMR porosities is producible liquid porosity.

FIG. 7. Data fitting results on time-lapse evolution of NMR porosity of the remaining fluid in rock sample. Track 1: Experimental data of time-lapse evolution of NMR porosity (black curve) and fitted data with equation 4 (grey curve); Track 2: Diffusivity distribution with cutoff (vertical line), with average diffusivity (dot) of producible water; Track 3: Immobile fluid porosity from time-lapse evolution of NMR porosity curve at time end (hollow circle) and from diffusivity distribution using cutoff (triangle).

FIG. 8. Implementation of algorithm based on non-negative matrix factorization (NMF) and hierarchical clustering to differentiate pore fluids based on their characteristic T₁-T₂ signatures. Plot (a) shows polygons from unsupervised learning; plot (b) shows fluid classification using polygons on T₁-T₂ map based on T₁/T₂ ratio and T₂; plot (c) presents a T₁-T₂ map of NMR log; and plot (d) displays implementation of fluid typing polygons on T₁-T₂ map for fluid porosities.

FIG. 9. Cross-plot of clay-bound water porosity from different core measurements as the function of clay measured from core XRD; and comparison of fluid porosity from core and log measurements. Plot (a) shows a cross-plot of clay-bound water porosity from different core measurement vs. clay measured from core XRD and plot (b) presents a plot of fluid porosity from NMR log vs. fluid porosity from multi-physics core measurements.

### DETAILED DESCRIPTION

GIP—Where the same core or half core is to be used in the workflow, gas-filled porosity is preferably measured prior to other measurements, but GIP and NMR order may be reversed if desired. Standard procedures are used to determine GIP.

NMR—In recent years, NMR 2D T₁-T₂ measurement has been widely used to differentiate pore fluid types encountered in tight reservoirs. In tight rock, it is observed that hydrocarbon-saturated rocks exhibit higher T₁/T₂ ratios than water-saturated rocks. In the T₁-T₂ map, fluid typing is performed based on T₁/T₂ ratio cutoffs. The water and hydrocarbon portions with different mobilities show different T₂ distributions, respectively. Combining T₁/T₂ ratio and T₂ cutoff lines, fluid type and mobility are identified using two rules: 1) hydrocarbon in tight rock has higher T₁/T₂ ratio than water; 2) producible fluid has longer T₂ than immobile fluid. If the T₁-T₂ signatures of different pore fluids are well identified and can be separated by T₁/T₂ and T₂ cutoff lines, fluid typing, and quantification can be achieved for different fluid porosities [212].

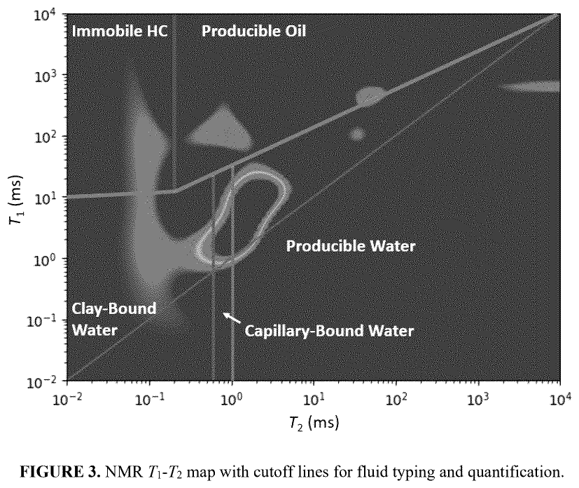

FIG. 3 shows an example of T₁-T₂ map from core NMR measurement on a tight rock sample. For this sample, the T₁-T₂ signatures of some pore fluids, such as clay-bound water, immobile hydrocarbon and producible oil are well separated by cutoff lines. The T₁-T₂ signatures of capillary-bound and producible water, form one peak on the map and become indistinguishable visually. The cutoff lines for fluid regions are often manually set and depend on laboratory measurements and interpreter's judgment, resulting in uncertainties of fluid porosity quantification. To obtain more accurate porosities by fluid type and mobility from core NMR interpretation, the integration and calibration using fluid porosities from other measurements are needed.

CLOSED RETORT—With closed retort analysis, the temperatures at which water and hydrocarbons are released from core samples classify these fluids by type and mobility (Handweger et al., 2011 and 2012). Released hydrocarbon is immiscible with water and can be collected and quantified separately.

For rock samples with little or no smectite [202], fluids with relatively higher mobilities, for instance, producible water residing in rock matrix pores (including capillary-bound and producible water), are released at lower temperature (Temp 1) [203].

Immobile fluids, such as clay-bound water, are released at higher temperature (Temp 2) [204]. At higher temperature (Temp 2), most of the rocks can release all the water, along with producible light oil [205].

The optimal temperature to separate clay-bound and matrix pore water (including producible water and capillary-bound water) in closed retort (temperature cutoff, Temp 1) was determined by increasing the temperature for the rock sample in multiple temperature steps within a temperature range, as shown in FIG. 3. The cutoff temperature was set at the minimum volume of the incrementally released water (see Temp 1 in FIG. 3).

Once the temperature cutoff was determined, a two-temperature heating protocol was applied to the sample-first to this optimal temperature (Temp 1) and subsequently to a higher temperature (Temp 2) wherein all producible fluids are released.

The two-temperature heating protocol usually works well except for smectite-rich rock [209]. When getting heated, smectite starts releasing its loosely-bound interlayer water (still part of the clay-bound water) at temperature lower than the boiling point of water (~100° C.), which is lower than the cutoff temperature for clay-bound and matrix pore water, causing underestimation of clay-bound water and overestimation of matrix pore water. Under this circumstance, porosity measurement of producible light oil at Temp 2 [210] is unaffected. However, only total amount of released water [211] can be still accurately quantified. Thus, in cores with significant amounts of smectite (≥5%), a single Temp 2 heating protocol is used, and immobile versus mobile water is determined using D₂O/H₂O exchange.

D₂O EXCHANGE—The H₂O/D₂O exchange experiment on core sample studies the diffusion of H₂O and D₂O molecules. Before the H₂O/D₂O exchange experiment, gas-filled porosity is measured followed by NMR T₁-T₂. Then the core sample gets submerged into bulk D₂O. D₂O will diffuse into the sample and H₂O will diffuse from the sample into the bulk liquid. The amount of remaining H₂O in the core sample is a function of diffusion time and the diffusivity of H₂O/D₂O in the core sample. Time-lapse evolution of remaining H₂O porosity in the core sample can be monitored using ¹H NMR since D₂O has no contribution to the measured NMR signal.

The NMR signal can be recorded using Carr-Purcell-Meiboom-Gill (CPMG) echo decay or free induction decay (FID) as the function of time. The recorded NMR signal is then calibrated and converted to NMR fluid porosity. In the H₂O/D₂O exchange experiment, what NMR measures is the signal of 1H atoms from both rock sample and the surrounding bulk fluid. Taking advantage of large contrast of T₂ relaxation time of 1H atoms inside the sample and in the bulk fluid, it is easy to separate the T₂ distribution from rock fluid and effluent. If the oil inside the rock is displaced by D₂O imbibition and enters into the bulk fluid, its NMR signal can also be detected as a separate T₂ peak. Thereby, the NMR signal of fluid inside rock sample is separated to get time-lapse evolution of rock pore fluids.

As H₂O diffuses out of the sample into the bulk fluid, the NMR porosity of remaining fluid in the core sample decreases with time. In the first few hours after rock sample immersed in D₂O, H₂O porosity reduces rapidly, followed by relatively slower change in the next 200 hours. To capture the initial rapid change of NMR porosity, the time step between the two concessive NMR measurements needs to be short enough. On the other hand, NMR acquisition time should be shorter than the time step. Because of the diffusion of H₂O/D₂O, the NMR properties of the fluids in the rock are averaged during the measurement.

To obtain more accurate data at each time point, the NMR acquisition time should be as short as possible. NMR measurement with CPMG or FID sequences are good methods to use due to their relatively short acquisition time. Compared to FID, CPMG measurement can get not only NMR porosity, but also T₂ distribution, giving more information. For high-frequency NMR, 2D T₁-T₂ measurement is also considered a suitable alternative, as long as the acquisition time is short enough.

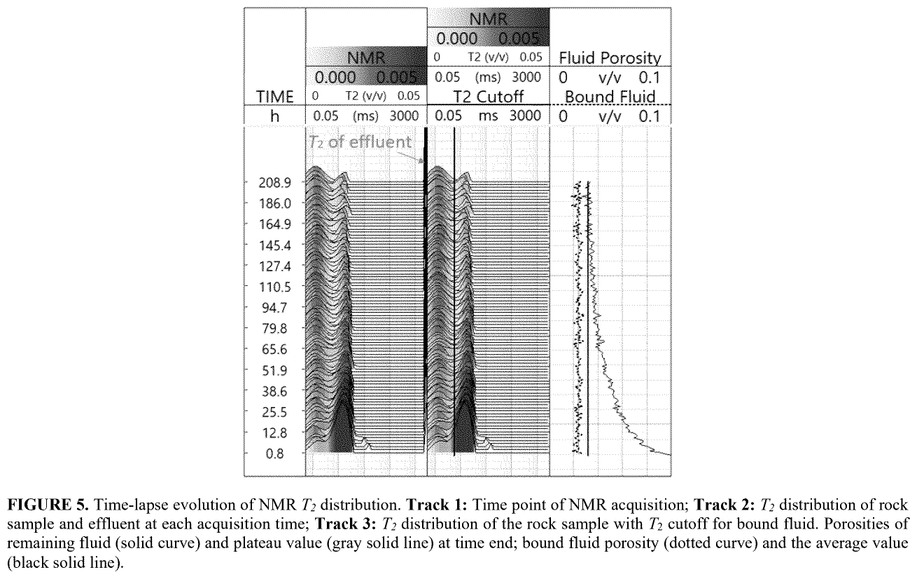

FIG. 5 shows an example of time-lapse NMR T₂ and porosity evolution from an NMR CPMG measurement. The first leftmost track gives the time point of each NMR acquisition. The second central track shows the T₂ distribution in the rock and effluent in the bulk at each NMR acquisition. The T₂ peak at the right end of the distribution is that of the effluent in the bulk. The third track on the right displays the T₂ distribution of remaining fluid in the rock by removing the T₂ peak of effluent. The T₂ of fluid in rock sample shows bimodal distribution with two peaks. The peak with shorter T₂ remains almost unchanged, representing signal from bound fluid (including clay-bound water and immobile hydrocarbon), while the peak with longer T₂ keeps decreasing magnitude with time. A T₂ cutoff can be set at the valley of the bimodal distribution to separate bound fluid from other pore fluids. The third track shows time-lapse evolution of remaining fluid porosity from NMR measurement and bound fluid porosity from T₂ distribution using the cutoff.

At the end of NMR acquisition, bound fluid porosity is lower than remaining fluid porosity. If the curve of remaining fluid porosity reaches a plateau, the plateau value is called end-time fluid porosity. The fluid remaining in the rock at time end include clay-bound and capillary-bound water, immobile hydrocarbon and some light oil. The discrepancy between end-time fluid porosity and bound fluid porosity gives porosities of capillary-bound water and light oil. During H₂O/D₂O, the major fluid change is H₂O/D₂O exchange. A portion of the light oil can be displaced by D₂O due to imbibition. Based on our observation, this occasionally happens but is not a problem since the released light oil is not miscible with D₂O and can be quantified easily, as described in [0087]. The water porosity from effluent is the producible water porosity [213].

After H₂O/D₂O exchange experiment, closed retort measurements at Temp 2 were performed to get porosities of immobile water and light oil. This is a one-temperature retort so it provides total immobile water, but cannot separate clay-bound and capillary-bound water. Immobile HC doesn't release at Temp 2 so we only get light oils.

TOTAL POROSITY—After H₂O/D₂O exchange experiment and closed retort measurement (where performed), crushed rock analysis of the sample is performed with solvent cleaning and drying to get grain volume and total porosity.

For half-core 1 the discrepancy between retort porosity and total porosity is the porosity of immobile hydrocarbon.

For half-core 2, after the D₂O exchange and one-step closed retort analysis at Temp 2 total porosity is determined. Crushed rock analysis is again conducted with solvent cleaning and drying to get grain volume and together with bulk volume the total porosity can be obtained. Immobile hydrocarbon porosity can be obtained using the same way discussed above. Bound fluid from H₂O/D₂O exchange including clay-bound water and immobile hydrocarbon. Bound fluid porosity (from H₂O/D₂O exchange experiment) subtracting immobile hydrocarbon porosity (from retort as described above) gives clay-bound water porosity. Capillary-bound water porosity can be calculated using time-end fluid porosity (from H₂O/D₂O exchange experiment) subtracting bound fluid porosity (from H₂O/D₂O exchange experiment) and light oil porosity (oil porosity from close retort combining the porosity of released light oil quantified earlier).

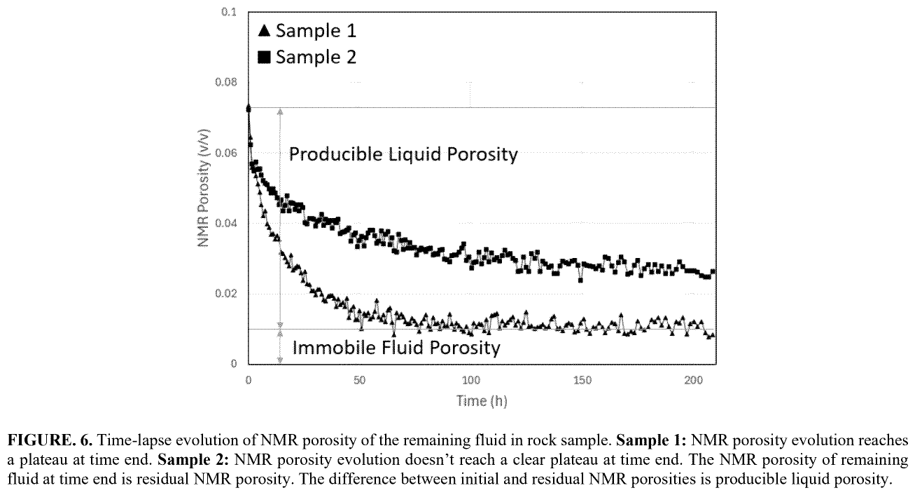

The evolution curve of remaining fluid porosity does not always reach a plateau. FIG. 6 shows two examples of time-lapse of measured NMR porosity of fluid in the rock sample. Since two half samples were analyzed and showed similar initial NMR fluid porosity in the rock sample, the duplicates provide good quality control. The diffusion of H₂O/D₂O in sample 1 is faster than sample 2. After enough time (e.g. 200 hours), the NMR porosity of sample 1 shows nearly no change. This method of immobile fluid estimation from evolution curve plateau is relatively straightforward, but some samples may have some uncertainty to difficulty in determining an accurate asymptote of the plateau (e.g. sample 2). At the end, the sample 2 curve still shows slowly decreasing NMR porosity, making it difficult to find the accurate plateau value for immobile fluid porosity.

Another way to quantify immobile and producible fluid porosities is using the diffusion model. When a cylindrical porous core sample with H₂O is immersed in bulk D₂O at time zero, the diffusion of H₂O/D₂O is transient. The amount of remaining H₂O in the sample is a function of diffusion time, sample dimensions and fluid diffusivity. The normalized NMR porosity of H₂O in the sample can be expressed as (Wang et al., 2021):

$$\begin{matrix}{MRP}^{N} = \frac{MRP ( t ) - MRP ( t = ∞ )}{MRP ( t = 0 ) - MRP ( t = ∞ )} \\ = \{ {\sum }_{n = 0}^{∞} \frac{8}{{( 2 n + 1 )}^{2} {π}^{2}} exp [ - \frac{{D ( 2 n + 1 )}^{2} {π}^{2}}{{L}^{2}} t ] \} \{ {\sum }_{n = 0}^{∞} \frac{4}{{α}_{n}^{2}} exp [ - \frac{4 D {α}_{n}^{2}}{{d}^{2}} t ] \}\end{matrix} \qquad ( 1 )$$

where MRP^{N} is the normalized NMR porosity of H₂O in the sample; MRP(t) is the NMR porosity of H₂O in the sample at time t; MRP(t=0) is the NMR porosity of H₂O in the sample at time zero; MRP(t=∞) is the NMR porosity of H₂O in the sample at infinite time; L is the length of the cylindrical sample; dis the diameter of the cylindrical sample; D is the diffusivity of H₂O/D₂O in the rock sample; and an are the positive roots of the Bessel function of the first type of order zero.

In equation (1), it is assumed the diffusion of H₂O/D₂O in the rock sample is homogeneous and a single value of diffusivity is used. If multiple values of diffusivity are considered to correspond to different pore throat sizes, equation (1) can be extended as follows:

$$\begin{matrix}{MRP}^{N} = \frac{MRP ( t ) - MRP ( t = ∞ )}{MRP ( t = 0 ) - MRP ( t = ∞ )} \\ = {\sum }_{i = 1}^{k} {f}_{i} \{ {\sum }_{n = 0}^{∞} \frac{8}{{( 2 n + 1 )}^{2} {π}^{2}} exp [ - \frac{{{D}_{i} ( 2 n + 1 )}^{2} {π}^{2}}{{L}^{2}} t ] \} \{ {\sum }_{n = 0}^{∞} \frac{4}{{α}_{n}^{2}} exp [ - \frac{4 {D}_{i} {α}_{n}^{2}}{{d}^{2}} t ] \}\end{matrix} \qquad ( 2 )$$

Here it is assumed the diffusion of H₂O/D₂O has a diffusivity distribution with k portions. fᵢ and Dᵢ represent the porosity and diffusivity of portion i.

MRP(t=∞) is the asymptote of the NMR porosity evolution curve. If the plateau of NMR porosity evolution curve is clear, the MRP(t=∞) can be considered to be equal to the plateau value of evolution curve (sample 1 curve as shown in FIG. 6). The normalized NMR porosity MRP^{N} is then calculated and used for data fitting with equation (2). If NMR porosity evolution curve does not reach a clear plateau and MRP(t=∞) cannot be easily determined (sample 2 curve as shown in FIG. 6), direct data fitting using equation (2) to get MRP(t=∞), MRP(t=0) and Dᵢ is not stable. In this case, a more general form of equation (2) can be expressed as equation (3) and (4):

$$MRP ( t ) = {\sum }_{i = 1}^{k} MRP ( t = 0 ) {f}_{i} \{ {\sum }_{n = 0}^{∞} \frac{8}{{( 2 n + 1 )}^{2} {π}^{2}} exp [ - \frac{{{D}_{i} ( 2 n + 1 )}^{2} {π}^{2}}{{L}^{2}} t ] \} \{ {\sum }_{n = 0}^{∞} \frac{4}{{α}_{n}^{2}} exp [ - \frac{4 {D}_{i} {α}_{n}^{2}}{{d}^{2}} ] \} \qquad ( 3 )$$

$$\begin{matrix}MRP ( t ) = {\sum }_{i = 1}^{k} {f}_{i}^{′} \{ {\sum }_{n = 0}^{∞} \frac{8}{{( 2 n + 1 )}^{2} {π}^{2}} \} exp [ - \frac{{{D}_{i} ( 2 n + 1 )}^{2} {π}^{2}}{{L}^{2}} t ] \} \{ {\sum }_{n = 0}^{∞} \frac{4}{{α}_{n}^{2}} exp [ - \frac{4 {D}_{i} {α}_{n}^{2}}{{d}^{2}} t ] \} \\ where {f}_{i}^{′} = MRP ( t = 0 ) \times {f}_{i}\end{matrix} , \qquad ( 4 )$$

Here it is assumed that all fluids, including immobile fluids (assuming very low diffusivity), can diffuse from sample to bulk fluids. Therefore, at infinite time, MRP(t=∞) is equal to zero. Data fitting on experimental data using equation (4) gives diffusivity distribution of water in the rock sample, including producible and immobile water.

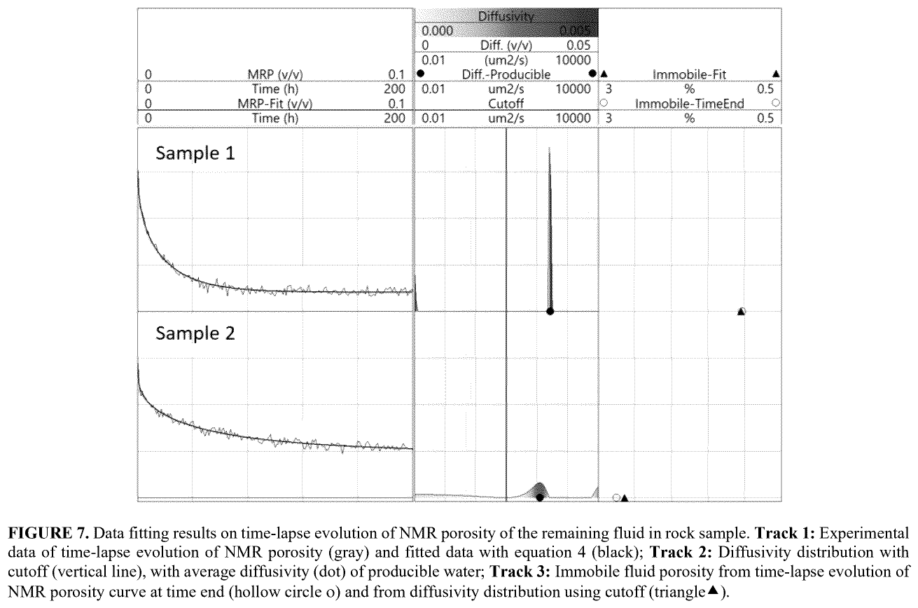

FIG. 7 shows data fitting results of time-lapse evolution of NMR porosity using the two samples from FIG. 6. A diffusivity distribution can be obtained from fitting results using equation (4). In FIG. 7, the first track on the left shows the data fitting results of time-lapse evolution of NMR porosity. The second central track shows diffusivity distribution of the two samples. The diffusivity distribution of sample 1 shows two sharp and distinct peaks. The peak at the left end represents the diffusivity of immobile fluid from data fitting, though immobile fluid may not actually diffuse. The second peak on the right shows the diffusivity of producible water. These two peaks are well-separated, and porosities of immobile fluid and producible water are calculated from these two peaks, respectively.

The diffusivity distribution of sample 2 also shows two peaks, but with larger standard deviation. A cut off can be carefully set at the valley of the bimodal distribution to separate two peaks for fluid porosities. The wider range of the distribution also suggests more pore structure heterogeneity of the rock.

The last track on the right displays immobile fluid porosities from time-lapse evolution of NMR porosity curve at time end (hollow circle), and from diffusivity distribution with diffusivity cutoff (triangle). For sample 1, immobile fluid porosities from two methods are very close. However, these porosities of sample 2 are different. This is because the time-lapse evolution of NMR porosity curve doesn't reach a plateau at time end, causing overestimation of immobile fluid porosity.

The diffusivity distribution from time-lapse evolution of NMR porosity not only gives more accurate immobile fluid porosities but also provides the average diffusivity of producible water. For water-saturated rock sample, there is a relationship between the average diffusivity of producible water and tortuosity of the rock, as shown as follows (Perkins et al., 1963):

$$τ = \frac{{D}_{bulk}}{D} \qquad ( 5 )$$

where D is the apparent diffusivity of water in the rock; and D_{bulk} is the diffusivity of bulk fluid.

For water in the rock with diffusivity distribution, equation (5) can be rewritten as follows:

$$τ = \frac{{D}_{bulk}}{{D}_{avg}} \qquad ( 6 )$$

where D_{avg} is the geometric mean value of diffusivity of producible water in the rock; and D_{bulk} is the diffusivity of bulk fluid.

The tortuosity of the rock can be integrated with producible fluid porosity and pore throat sizes (e.g. from MICP) to get permeability of the rock (Wang et al., 2021):

$$k = \frac{{d}_{rms}^{2} {∅}_{producible}}{32 τ} \qquad ( 7 )$$

Here dᵣₘₛ is square root of the arithmetic mean of the squares of pore throat sizes; Φ_{producible} is producible fluid porosity.

INTEGRATION—when all the analyses are complete, we integrate the various data using multiphysics simulations. Any software package can be used, but herein we used internal proprietary program. Fluid porosities from the various benchtop core measurements are integrated and cross-validated to get final porosity answers of immobile water and hydrocarbon, producible water and light oil. For instance, fluid porosities from close retort and H₂O/D₂O exchange experiment can be used to calibrate the cutoff line for core NMR fluid typing and quantification using T₁-T₂ map, as described above. S2 from Rock-Eval TOC pyrolysis is also used as reference in comparison to immobile hydrocarbon porosity.

Nevertheless, core data are discrete and usually can't cover the whole interval of interest. Moreover, except for pressure-preserved core, core samples are usually missing light end of hydrocarbon (producible) and partial amounts of producible water due to gas expansion effect (or pore pressure reduction effect) when the core barrel reaches the surface. Thus, to obtain more accurate and continuous in-situ fluid porosities, especially producible fluid porosities, NMR 2D T₁-T₂ depth logs are integrated and calibrated using the answers from the various core measurements described herein.

Compared to core NMR, NMR logging data has much lower signal-to-noise ratio, causing low resolution of T₁-T₂ map and the signatures of individual fluid components are generally not well-separated. These complicate the application of fluid characterization methods using cutoff lines as it is challenging to visually differentiate the NMR signal contributions of different formation fluids in T₁-T₂ maps.

Recently, an automated unsupervised learning algorithm based on non-negative matrix factorization (NMF) and hierarchical clustering was developed to optimize the signal separation in NMR T₁-T₂ maps (Venkataramanan, 2018). In the workflow described herein, we have implemented this algorithm to differentiate pore fluids based on their characteristic T₁-T₂ signatures. This method first deconvolves the T₁-T₂ maps of different depths from the same formation or litho-facies to extract T₁-T₂ maps of common individual fluid using NMF, followed by hierarchical clustering to get the number of clusters and the polygon boundaries between clusters.

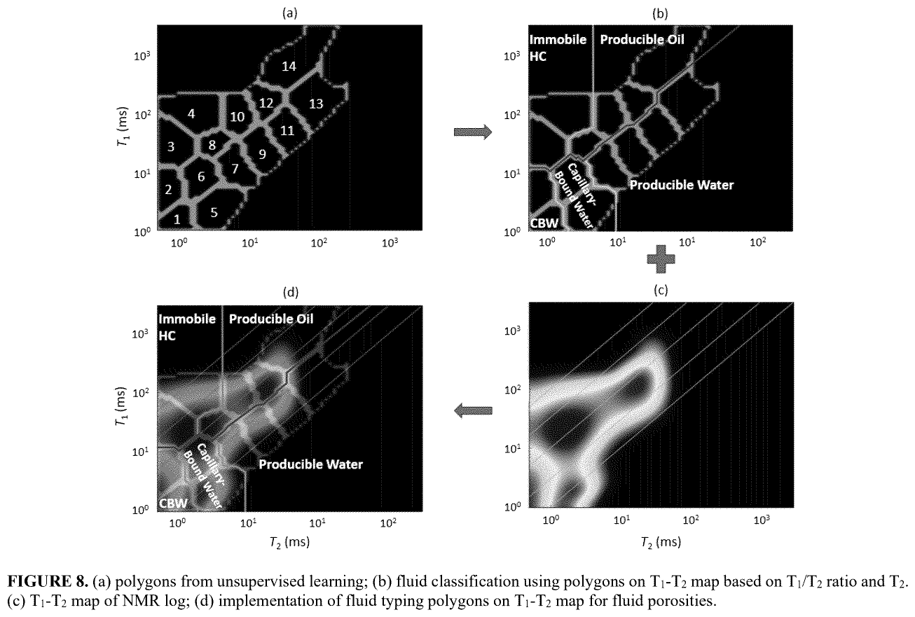

FIG. 8 shows four plots to exemplify differentiation of pore fluids based on their characteristic T₁-T₂ signatures. Plot (a) shows an example of clustering result from T₁-T₂ maps of NMR depth log in a zone of interest. Unsupervised learning result itself cannot classify fluid types. Fluid typing and mobility identification of each cluster need be manually set by interpreter based on the T₁-T₂ ratio and T₂ of each cluster, as shown in plot (b). Fluid typing polygons are then implemented on T₁-T₂ map shown in plot (c) for fluid porosities as shown in plot (d). This manual fluid classification and quantification may result in uncertainties of fluid typing and porosities.

By integration with fluid porosity from multi-physics core measurement, a 2-step fluid tagging and porosity quantification is developed to get more accurate fluid porosities. In the first step, fluid type of each cluster is tagged using the same rules as described above, namely: 1) hydrocarbon in tight rock has higher T₁/T₂ ratio than water; 2) producible fluid has longer T₂ than immobile fluid. By the rules, cluster in FIG. 8 plot (a) is tagged as clay-bound water and it is not possible to be producible oil. However, for the fluid cluster close to the fluid region boundary, such as cluster 8 on the map, could be either water or hydrocarbon, bound or producible.

To reduce the uncertainty of fluid tagging on such clusters, step 2 is introduced in a more quantitative way. The cost function is set to minimize the sum of fluid porosity differences of all the fluids after fluid tagging, as shown below:

$$g = min \{ {\sum }_{i = 0}^{m} {[ {\sum }_{j = 1}^{n} {( {∅}_{j , core} - {\sum }_{k ∈ j} {∅}_{k} )}^{2} ]}_{i} \} \qquad [ 8 ]$$

Here n is the number of fluids used in this step and m is the number of core data points with outliers excluded. Each cluster k is tagged as fluid j at each core data point i. The fluid tagging of boundary cluster is optimized to minimize the cost function.

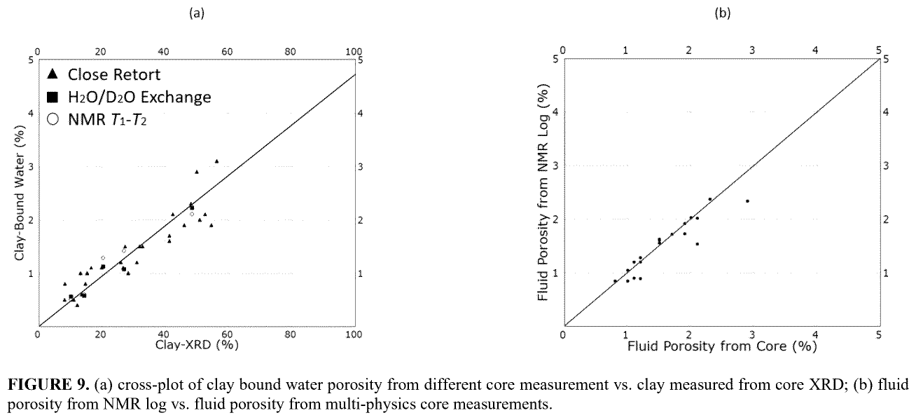

FIG. 9 shows two plots (a) and (b). Plot (a) gives a cross-plot of clay-bound water porosity from different core measurements (close retort analysis, H₂O/D₂O exchange experiment and NMR T₁-T₂ measurement) as the function of clay measured from core XRD. The porosities from different core measurements are consistent each other and show good correlation to the amount clay measured by core XRD (trend line in black). Plot (b) in FIG. 9 displays the comparison of fluid porosity from core and log measurements. The data shows a good match (1:1 line in black), confirming the validity of our methodology.

The following references are each incorporated by reference in their entirety for all purposes.
Burnett A D., A quantitative X-Ray diffraction technique for analyzing sedimentary rocks and soils, J. Test. Eval. March 23(2): 111-118 (1995).
Carman, P. C., Fluid flow through granular beds, Process Safety and Environmental Protection: Transactions of the Institution of Chemical Engineers, Part B., 75, S32-S48 (1997).
Dick M. J.; Veselinovic D.; Green D; Review of recent developments in NMR core analysis, Petrophysics, 63 (03): 454-484, (2022).
Handwerger D. A., et al., Improved petrophysical core measurements on tight shale reservoirs using retort and crushed samples, SPE, 147456-MS (2011).
Handwerger D. A., et al., Reconciling retort versus Dean-Stark measurements on tight shales, SPE, 159976-MS (2011).
Jiang, T.; et al., Integrated reservoir characterization using unsupervised learning on Nuclear Magnetic Resonance (NMR) T1-T2 Logs, SPWLA-5047 (2020).
Kausik R. et. al., NMR Relaxometry in Shale and Implications for Logging, SPWLA 56th Annual Logging Symposium, July 18-22, SPWLA-2015-SSSS (2015).
petrowiki.spe.org/Porosity_for_resource_in_place_calculations
Perkins J.; Johnston O. C., A Review of Diffusion and Dispersion in Porous Media, SPE-480-A (1963).
Recommended practices for core analysis, Second edition, Chapter 4, February 1998, American Petroleum Institute. Available online at energistics.org/sites/default/files/2022-10/rp40.pdf
Soil quality—Determination of organic and total carbon after dry combustion (elementary analysis), ISO 10694:1995.
Standard practice for general techniques for obtaining infrared spectra for qualitative analysis, ASTM E1252-98.
Sun, J., et al., Measurement of total porosity for gas shales by gas injection porosimetry (GIP) method, Fuel 186:694-707 (2016), available online at doi.org/10.1016/j.fuel.2016.09.010.
Venkataramanan J., et al., An unsupervised learning algorithm to compute fluid volumes from NMR T₁-T₂ logs in unconventional reservoirs, 2018, SPWLA-2018-Y.
Wang X., et al., Permeability from NMR in the unconventional point-pleasant formation, Unconventional Resources Technology Conference (URTeC), Houston, Texas, USA, July 26-28, URTeC-5015 (2021).
wiki.aapg.org/LECO_method_in_estimating_richness
petrowiki.spe.org/NMR_petrophysics
wiki.aapg.org/Rock-Eval_pyrolysis
wiki.aapg.org/Porosity
Zumberge, A. J., et al., Fluid definitions and pore space partitioning: Integrating NMR and closed retort data, SPE/AAPG/SEG Unconventional Resources Technology Conference, URTEC-3863806-MS (2023).

1) A method comprising:
a) obtaining a sample of core from a reservoir;
b) acquiring simultaneous nuclear magnetic resonance (NMR) relaxation time in longitudinal (T₁) and transverse (T₂) directions (NMR T₁-T₂) on a portion of said core to determine an amount of matrix pore water and a volume of clay-bound water;
c) soaking a portion of said core in D₂O;
d) performing time-lapse NMR on H₂O/D₂O exchange in said D₂O soaked core to quality check said NMR T₁-T₂, wherein said H₂O/D₂O exchange eliminates a matrix pore water signal, leaving a clay-bound water signal; and
e) using said clay-bound water signal from step d) to correct said volume of clay-bound water from step b).

2) The method of claim 1, further comprising obtaining gas injection porosity from a portion of said core.

3) The method of claim 1, further comprising obtaining total organic carbon and/or rock eval pyrolysis data from a portion of said core.

4) The method of claim 1, further comprising crushing, cleaning and drying a portion of said core or said trimmings to obtain total porosity of said core.

5) The method of claim 1, further comprising:
obtaining closed retort data from a portion of said core at Temp 1 and Temp 2, or at Temp 2 if said core contains smectite, wherein Temp 1 is a temperature where mobile water is emitted from said core and Temp 2 is a temperature where bound water and light oils are emitted from said core.

6) The method of claim 5, further comprising:
integrating core data to separate and quantify a volume of producible water, capillary-bound water and clay-bound water, and to separate and quantify a volume of producible hydrocarbons and immobile hydrocarbons.

7) The method of claim 6, further comprising determining a bulk volume of a portion of said core and using said bulk volume to convert volume to porosity.

8) A method comprising:
a) obtaining a sample of core from a reservoir;
b) trimming said core to produce trimmings and a remnant of core (hereinafter core);
c) obtaining gas injection porosity from a portion of said core;
d) acquiring simultaneous nuclear magnetic resonance (NMR) relaxation time in longitudinal (T₁) and transverse (T₂) directions (NMR T₁-T₂) from a portion of said core;
e) obtaining core minerology data from said trimmings to determine a level of smectite in said core and performing steps f) and i) if said trimmings have 0 to <5% smectite, but performing steps g), h), and i) if said trimmings have ≥5% smectite;
f) obtaining closed retort data from a portion of said core at Temp 1 and Temp 2, wherein Temp 1 is a temperature where mobile water is emitted from said core and Temp 2 is a temperature where bound water and light oils are emitted from said core; or
g) soaking a portion of said core in D₂O and performing time-lapse NMR on H₂O/D₂O exchange in said D₂O soaked core to separate and quantify producible water from immobile water;
h) obtaining closed retort data on a portion of said core at Temp 2; and
i) integrating core data to separate and quantify a volume of producible water, capillary-bound water and clay-bound water, and to separate and quantify a volume of producible hydrocarbons and immobile hydrocarbons.

9) The method of claim 8, further comprising determining a bulk volume of said core and using said bulk volume to convert volume to porosity.

10) A method comprising:
a) obtaining a sample of core from a reservoir;
b) trimming said core to produce trimmings and a remnant of core (hereinafter core);
c) obtaining gas injection porosity from a portion of said core;
d) acquiring simultaneous nuclear magnetic resonance (NMR) relaxation time in longitudinal (T₁) and transverse (T₂) directions (NMR T₁-T₂) from a portion of said core;
e) obtaining total organic carbon and/or rock eval pyrolysis data from a portion said trimmings;
f) obtaining core minerology data from a portion of said trimmings to determine a level of smectite in said core and performing steps g), j) and k) if said trimmings have 0 to <5% smectite, but performing steps h), i), j) and k) if said trimmings have ≥5% smectite;
g) obtaining closed retort data from a portion of said core at Temp 1 and Temp 2, wherein Temp 1 is a temperature where mobile water is emitted from said core and Temp 2 is a temperature where bound water and light ends are emitted from said core;
h) soaking a portion of said core in D₂O and performing time-lapse NMR on H₂O/D₂O exchange in said D₂O soaked core to separate and quantify producible water from immobile water;
i) obtaining closed retort data on a portion of said core at Temp 2;
j) crushing, cleaning and drying a portion of said core or said trimmings to obtain total porosity data; and
k) integrating data using multiphysics simulations obtained from any performed steps from c) to j) to separate and quantify a volume of immobile water and producible water and a volume of immobile hydrocarbons and producible hydrocarbons.

11) The method of claim 10, further comprising determining a bulk volume of said core and using said bulk volume to convert volume to porosity.

12) A method comprising:
a) obtaining a sample of core from a reservoir;
b) trimming said core to produce trimmings and a remnant of core and dividing said core into at least half-1 and half-2;
c) obtaining core minerology data from a first portion of said trimmings to determine a level of smectite in said core and obtaining Rock-Eval total organic carbon (TOC) from a second portion of said trimmings;
d) obtaining gas filled porosity from said half-1 and said half 2 to obtain a gas volume in said core;
e) acquiring simultaneous nuclear magnetic resonance (NMR) relaxation time in longitudinal (T₁) and transverse (T₂) directions (NMR T₁-T₂) from said half-1 and said half 2 to quantify volume(s) of one or more of producible water, capillary-bound water, clay-bound water, producible hydrocarbons, and immobile hydrocarbons;
f) obtaining closed retort data on said half-1 to quantify volume(s) of one or more of producible water, capillary-bound water, clay-bound water, and producible hydrocarbons:
i) at Temp 1 and Temp 2 if said core has no smectite, wherein Temp 1 is a temperature where producible water and capillary-bound water are emitted from said core and Temp 2 is a temperature where clay-bound water and light ends are emitted from said core; or
ii) at Temp 2 if said core has significant smectite (≥5%);
g) soaking said half-2 in D₂O and performing time-lapse NMR on H₂O/D₂O exchange in said D₂O soaked half-2 to obtain a volume of clay-bound water;
h) obtaining closed retort data at Temp 2 on said soaked half-2 to obtain a volume of total water and a volume of producible oil;
i) crushing, cleaning and drying a portion of said half-1 from step f) and said soaked core half-2 from step h) to obtain a total fluids volume in said core;
j) integrating half-1 and half-2 data from steps d) through i) using multiphysics simulations to separate and quantify a volume of: producible water, capillary-bound water, and clay-bound water and to separate and quantify a volume of immobile hydrocarbons and producible hydrocarbons and using S2 from said Rock-Eval TOC as reference in comparison to immobile hydrocarbon porosity.

13) The method of claim 12, further comprising determining a bulk volume of said core and using said bulk volume to convert volume to porosity.
